(19) United States
(12) Patent Application Publication
(10) Pub. No.: US 2026/0083148 A1
(43) Pub. Date: Mar. 26, 2026

(54) KOMBUCHA FERMENTED WITH MONOSACCHARIDES AND HAVING A REDUCED ALCOHOL CONTENT

(71) Applicant: Filipp Mirzakhanov, Centennial, CO (US)

(72) Inventor: Filipp Mirzakhanov, Centennial, CO (US)

(21) Appl. No.: 19/333,564

(22) Filed: Sep. 19, 2025

Related U.S. Application Data
(60) Provisional application No. 63/698,464, filed on Sep. 24, 2024.

(51) Int. Cl. A23F 3/16; A23F 3/40; A61K 31/4375; A61K 36/74

(52) U.S. Cl. CPC A23F 3/166; A23F 3/405; A61K 31/4375; A61K 36/74

(57) ABSTRACT

The present invention produces fermented kombucha beverages with reduced alcohol content by utilizing allulose or analogues thereof as the primary fermentable sugar. These rare sugars limit ethanol production during fermentation while preserving flavor, probiotic benefits, and acidity. The invention further encompasses the incorporation of Kratom (Mitragyna speciosa) extracts or alkaloids into the fermentation process or final product, enabling a low-alcohol kombucha with potential enhanced wellness properties. The resulting beverage maintains an alcohol content below 0.5% by volume, making it suitable for non-alcoholic labeling and broader consumer appeal. The invention is preferably a bottled beverage.

### FIELD OF THE INVENTION

Methods of SCOBY fermentation for Kombucha and other beverages having a low alcohol concentration (below 0.5%) by using monosaccharides including epimers and stereoisomers of fructose.

### BACKGROUND OF THE INVENTION

Kombucha is a fermented beverage produced from tea, sugar, and a symbiotic culture of bacteria and yeast (SCOBY). During the fermentation process, the yeast converts sugar into alcohol, which is subsequently converted into acetic acid by bacteria, giving kombucha its characteristic tangy flavor. However, this process can result in the production of ethanol, often leading to alcohol content in kombucha exceeding 0.5% by volume, which is a threshold for labeling the beverage as “non-alcoholic” in various jurisdictions.

Kombucha, water kefir, and similar fermented beverages are traditionally produced using cultures grown on botanical extracts sweetened with conventional sugars like sucrose, fructose, honey, maple syrup, or agave syrup. While these sugars are effective in promoting fermentation, they result in beverages that contain higher calorie content, higher glycemic indexes, and often a higher starting alcohol content.

Recent consumer trends favor beverages that are lower in calories, glycemic index, and alcohol content, while still providing the functional health benefits of probiotics and prebiotics. Naturally occurring rare sugars, such as allulose, tagatose, sorbinose, arabinose, trehalose, and isomaltulose, offer a solution. These sugars possess similar sweetness to conventional sugars but provide fewer calories, a lower glycemic response, and in some cases, prebiotic benefits.

The ability to reduce the alcohol content in kombucha while preserving its flavor and probiotic properties is desirable for regulatory compliance and to meet consumer preferences. Current methods of reducing alcohol content, such as pasteurization or filtration, can harm the integrity of the probiotic cultures or alter the flavor profile. Thus, a need exists for a method to naturally limit ethanol production during fermentation without compromising the quality of the kombucha.

U.S. Pat. No. 11,918,020 assigned to AquaCultured Foods, Inc. by Anne Palermo, and Robert Schultz, describes utilizing monosaccharides to optimize growth of a scoby colony used for producing protein in the form of a meat analogue. However, this patent does not describe the optimal ways of reducing alcohol content in any fermentation process.

U.S. Patent Publication No. 2023/0276830 by Filip Mirzakhonov describes a kombucha product including Kratom derived alkaloids, and such product recites a fermentation method that can be utilized with added fructose and glucose.

### SUMMARY OF THE INVENTION

The present invention provides a novel method for fermenting kombucha SCOBY, water kefir cultures, and other similar cultures on botanical extracts sweetened with rare sugars or combinations thereof. These rare sugars include, but are not limited to, allulose, tagatose, sorbinose, arabinose, trehalose, and isomaltulose, which may be used individually or in combination with each other or with conventional sweeteners such as sucrose, honey, and agave syrup, at concentrations ranging from 5 mg/mL to 500 mg/mL.

The resulting fermentation process produces a beverage with significantly lower calories, glycemic index, and alcohol content compared to traditional fermentation processes using conventional sweeteners.

Low-Calorie Fermentation is a feature of the invention. The rare sugars used contribute fewer calories than conventional sweeteners, resulting in a lower-calorie beverage. This makes the product more suitable for consumers seeking low-calorie options without sacrificing sweetness.

Low Glycemic Index is a result of the product of the present invention.

Fermentation of the culture on botanical extracts sweetened with allulose, tagatose, and other rare sugars results in a beverage with a reduced glycemic index compared to those fermented with conventional sweeteners. This is particularly beneficial for consumers managing blood sugar levels, such as those with diabetes.

Low Alcohol Content is a desirable attribute of the present invention as it avoids regulations imposed on alcohol-containing beverages. Many consumers prefer pro-biotic beverages that lack a bioactive alcoholic content. Low alcohol (or near zero) is preferred by recovering alcoholics, particularly when combined with a bioactive ingredient such as kratom.

The fermentation process using rare sugars such as allulose and tagatose results in beverages with a lower starting alcohol content compared to those produced using conventional sugars. This makes the product more appealing to consumers seeking beverages with minimal or no alcohol content.

The present invention provides many prebiotic and pro-biotic benefits to consumers.

Some rare sugars, particularly tagatose, have prebiotic properties that promote gut health. Fermenting cultures on these sugars enhances the prebiotic content of the resulting beverage, adding further health benefits. Probiotic benefit comes as the fermenting cultures are live in the final kombucha product. The pre-biotic sugars and probiotic cultures cooperate to optimize fermentation and to optimize gut health of the consumer.

The present invention provides a method for reducing the alcohol content in kombucha by introducing monosaccharides as the primary fermentable sugar. This produces kombucha with alcohol content below 0.5% by volume while preserving the essential characteristics of the beverage, including its flavor, probiotics, and acidity.

In one embodiment, the method includes replacing or reducing disaccharides. This occurs by replacing sucrose, with monosaccharides, such as glucose or fructose in a kombucha recipe. By using monosaccharides, the fermentation process is altered to favor reduced ethanol production while still allowing sufficient microbial activity to produce the desirable flavor and acidity.

The process described herein ensures the alcohol (ethanol) content remains under 0.5% by volume, thus rendering the kombucha non-alcoholic according to industry standards.

It can be appreciated that the present invention can also speed fermentation time due to use of the rare sugars i.e. monomers and analogues thereto. Various ingredients can be combined prior to or after fermentation. Two fermentation processes can be used, the second being post bottling. The present invention include a bottled kombucha, kefir, or other fermented beverage.

### DETAILED DESCRIPTION

In one embodiment of the invention, the fermenting kombucha SCOBY, water kefir, or other similar cultures using botanical extracts sweetened with one or more rare sugars, including but not limited to allulose, tagatose, sorbinose, arabinose, trehalose, and isomaltulose. These sugars can be used alone or in combination with each other or with conventional sweeteners at concentrations ranging from 5 mg/mL to 500 mg/mL.

The present invention is directed to a method of producing kombucha with reduced alcohol content, specifically to less than 0.5% by volume, by using monosaccharides during the fermentation process.

### Ingredients and Initial Preparation:

Tea: Brewed tea serves as the base of kombucha. Traditional tea varieties, such as black or green tea, may be used. In an alternative embodiment, Kratom tea is used instead of, or in conjunction with the brewed tea.

Monosaccharides are utilized instead of or in combination with disaccharides like sucrose. Preferably, specific monosaccharides such as isomers, stereoisomers, or epimers of glucose, fructose, or mixtures thereof are added to the brewed tea. These monosaccharides serve as the fermentable sugar source for the SCOBY. It can be appreciated that although the preferred embodiment of the invention yields kombucha, the present invention can be used to manufacture many foods, including protein rich meat analogues, and other engineered and nutritive foods.

Monosaccharides are the simplest form of carbohydrates, consisting of a single sugar unit. They cannot be hydrolyzed into simpler sugars. Common examples include glucose, fructose, and galactose.

Tagatose is also a monosaccharide and is structurally similar to fructose, but it is a stereoisomer, meaning it has the same chemical formula but a different arrangement of atoms.

Allulose (also known as D-psicose) is also a monosaccharide and is an epimer of fructose. It has a similar structure to fructose but differs slightly in the orientation of a hydroxyl group at one position.

Despite being monosaccharides, both tagatose and allulose have unique metabolic pathways that lead to low caloric content and minimal impact on blood glucose levels in vivo. These monosaccharides also have desirably sweet flavor profiles.

Galactose could technically be used as a substitute for tagatose or allulose in certain applications because it is also a monosaccharide and shares some similar properties. However, there are important difference is that it is not as sweet.

Yeasts (such as Saccharomyces cerevisiae, commonly used in brewing and baking) can ferment galactose, but this sugar is not their preferred substrate. Yeasts generally favor glucose or fructose for fermentation because these sugars are metabolized more efficiently.

Some yeasts have the necessary enzymes to break down galactose through the Leloir pathway, which converts galactose into glucose-1-phosphate, allowing it to enter the glycolysis pathway and be fermented into ethanol. This process is slower and less efficient compared to glucose fermentation, which makes it viable for the present invention.

The fermentation of galactose into alcohol typically occurs more slowly than the fermentation of glucose or fructose. This is because the yeast must first convert galactose into a form that can be processed through glycolysis, which adds an extra step and takes time.

The step of glycolysis is an important part of the method of various embodiments of the present invention as glycolysis is intended to slow any fermentation processes to yield a less than 0.5% alcohol content in the kombucha product.

In mixtures containing both galactose and glucose, yeasts will preferentially ferment glucose first, and only after the glucose is depleted will they begin metabolizing galactose. Preferably, no glucose is added to the fermentation, or a minimal amount of glucose is added but does not boost alcohol content of the kombucha product significantly.

While galactose can be fermented into alcohol, it is not typically used as a primary sugar source for brewing beer or making wine because of its slower fermentation rate and the preference of yeasts for other sugars like glucose and fructose.

Galactose is found in lactose (the sugar in milk), which is a disaccharide composed of glucose and galactose. When lactose is broken down, the galactose portion can be fermented by specific microorganisms, which is a key process in the production of yogurt or kefir, although ethanol production is minimal in these contexts. Galactose can also be used to produce kombucha with a minimal alcohol content.

In summary, galactose can be fermented into alcohol, but it is a slower and less efficient process compared to other sugars like glucose or fructose. This quality makes it viable for kombucha production in accord with the present invention.

There are differences between Galactose, Tagatose, and Allulose. Sweetness is one difference. Tagatose is about 92% as sweet as sucrose. Allulose is about 70% as sweet as sucrose. Galactose is Less sweet, approximately 30% as sweet as sucrose. This means that if one would substitute galactose, one would need more of it to achieve the same sweetness.

Tagatose has approximately 1.5 calories per gram, and allulose has close to 0.2 to 0.4 calories per gram. Galactose has about 4 calories per gram, the same as regular sugar (sucrose). Using galactose as a substitute would not offer the low-calorie benefits that tagatose and allulose provide.

Tagatose and allulose have minimal impacts on blood glucose levels and are ideal for people with diabetes or those following low-carb diets. Galactose is metabolized more like glucose and can raise blood sugar levels, making it less desirable for use in low-glycemic or diabetic-friendly products.

### Metabolic Pathways of Allulose and Tagatose in Humans

Allulose (D-psicose) and tagatose (D-tagatose) are rare monosaccharides classified as ketohexoses, structurally similar to fructose but with distinct metabolic fates that contribute to their low caloric value and minimal impact on blood glucose levels. Unlike common sugars like glucose or fructose, which are readily metabolized for energy, these sugars undergo limited metabolism in humans, making them attractive as low-calorie sweeteners. Below, I detail their metabolic pathways based on scientific reviews, including absorption, metabolism, and excretion. These pathways explain their health benefits, such as potential roles in managing obesity, diabetes, and gut health.

Allulose is absorbed, distributed, and excreted with minimal metabolism, resulting in a near-zero energetic yield (approximately 0.2-0.4 kcal/g). Allulose is absorbed in the small intestine via passive diffusion and facilitated transport. It primarily uses the GLUT5 transporter on the apical membrane of epithelial cells (with lower affinity than fructose) and GLUT2 for export across the basolateral membrane. The overall absorption rate is 50-70% of an oral dose, similar to fructose's limited absorption capacity. Doses above 0.4 g/kg body weight may cause intestinal discomfort due to osmotic effects from unabsorbed portions reaching the colon.

Unlike glucose, which enhances fructose absorption via GLUT2 recruitment, it's unclear if glucose similarly affects allulose.

After absorption, allulose enters the bloodstream and is rapidly cleared (half-life <15 minutes in animal models). It accumulates primarily in the liver and urinary bladder. In humans, postprandial plasma levels peak at around 1 hour and remain elevated for over 6 hours after a 0.5 g/kg dose, higher than fructose due to less gut metabolism.

The human genome seems to lack enzymes to metabolize allulose effectively, leading to negligible breakdown in human cells (unlike fructose, which is phosphorylated by ketohexokinase and enters glycolysis). It is highly stable in gastric and intestinal fluids, as well as hepatocytes, with no significant hepatic energy production. A small fraction may be decomposed into short-chain fatty acids (SCFAs) by cecal microbes in the large intestine, but overall fermentation is low (no significant breath hydrogen increase at doses up to 0.33 g/kg). Certain bacteria (e.g., Klebsiella pneumoniae) can utilize allulose in vitro, but human rates are minimal.

Allulose contrasts with fructose, which is extensively metabolized in the liver via the fructolytic pathway. Allulose cannot be metabolized effectively via this fructolytic pathway and is thus preferred to manage glycemic index of the product of the present invention.

The majority (50-70% of absorbed dose of allulose is excreted unchanged in urine via renal filtration. Some is also excreted in feces after microbial action in the colon, and in humans, it can also be discharged through sweat. This rapid excretion contributes to its low caloric impact.

Allulose's pathway supports its use in diabetes management by avoiding blood glucose spikes and potentially stimulating GLP-1 secretion from enteroendocrine cells, similar to fructose.

Tagatose is partially absorbed and metabolized, with a caloric value of about 1.5 kcal/g, primarily due to hepatic processing and gut fermentation. Approximately 20-25% of ingested tagatose is absorbed in the small intestine via passive diffusion, leaving 75-80% unabsorbed to reach the colon. Absorption is slower and less efficient than fructose, contributing to lower glycemic effects.

The absorbed fraction enters the portal vein and is transported to the liver, where it is principally metabolized. Distribution data in humans are limited, but it follows a pattern similar to fructose.

The 20% absorbed tagatose is fully metabolized in the liver. The unabsorbed 75-80% is fermented in the large intestine (caecum and colon) by gut microbiota, producing SCFAs without significant nutrient absorption interference. No metabolism occurs in the stomach or small intestine. Tagatose acts as a prebiotic, promoting beneficial bacteria growth. Metabolized portions contribute to energy via gluconeogenesis, but unabsorbed tagatose is fermented and excreted as gases (e.g., hydrogen, leading to flatulence at high doses) or in feces. No significant renal excretion is noted for the absorbed fraction.

Tagatose's pathway supports antidiabetic effects by reducing postprandial hyperglycemia and hyperinsulinemia, with additional benefits like weight loss and increased HDL cholesterol.

Comparison of Allulose and Tagatose Pathways is shown in Table 1 below.

TABLE 1

| Aspect | Allulose | Tagatose |
|---|---|---|
| Absorption | 50-70% via GLUT5/ | 20-25% via passive diffusion |
| | GLUT2 (similar | (slower than fructose) |
| | to fructose) | |
| Metabolism | Negligible in human cells; | 20% in liver like fructose |
| | no specific enzymes; minor | (e.g., to tagatose-1- |
| | gut fermentation | phosphate, gluconeogenesis); |
| | | 75-80% fermented in gut |
| Key | No hepatic metabolism; | Slower phosphorylation; more |
| Differences | lower fermentation | potent uric acid inducer |
| from | | |
| Fructose | | |
| Excretion | Mostly renal (urine); | Fermented portions as gases/ |
| | some fecal/sweat | feces; metabolized portions |
| | | via energy pathways |
| Caloric | 0.2-0.4 kcal/g (minimal) | 1.5 kcal/g (partial hepatic + |
| Yield | | gut fermentation) |
| Health | No glucose/insulin rise; | Low glycemic; prebiotic; |
| Impacts | potential GLP-1 stimulation | weight loss/HDL benefits |

Both sugars share fructose-like absorption but diverge in metabolism: allulose is largely inert and excreted, while tagatose is partially utilized like a slower fructose analog. These properties make them suitable for low-calorie applications, though high doses can cause GI side effects due to unabsorbed fractions.

While tagatose and allulose are only partially absorbed by the body and are largely excreted or fermented in the gut, contributing to their low-caloric nature, galactose is more readily absorbed and converted into glucose in the liver, making it more calorically dense and more likely to impact blood sugar levels.

Tagatose and allulose provide desirable functional properties like browning (Maillard reaction), good solubility, and texture enhancement.

Galactose does not share all of these properties to the same extent, especially regarding browning and texture in baked goods.

An epimer is a type of stereoisomer where two compounds differ in the configuration of atoms around only one specific carbon atom in a sugar molecule, while the rest of the molecule remains identical. This distinction occurs in molecules that have more than one chiral (asymmetric) carbon atom.

Key Points about Epimers:

Epimers are stereoisomers, meaning they have the same molecular formula and sequence of bonded atoms, but they differ in the three-dimensional orientation of atoms at one specific carbon atom. This occurs at a chiral carbon, which is a carbon atom attached to four different groups.

A benefit of utilizing the above-mentioned epimers of glucose or fructose is that they typically ferment more slowly than glucose because epimerization or glycolysis often precedes fermentation with most common yeast-based fermentation processes.

Glucose and galactose are epimers of each other. They differ only at the carbon atom C-4. In glucose, the hydroxyl group (—OH) on C-4 is on the right, while in galactose, it is on the left.

Glucose and mannose are also epimers, differing only at carbon atom C-2. Mannose can be used as a fermentation substrate in accordance with the present invention because mannose can take longer to ferment than common sugars like glucose or fructose. In most fermentation processes mannose must undergo epimerization into glucose. Saccharomyces yeast, lactobacillus bacteria, and Escherichia coli are known to be able to ferment mannose.

### Example of an Epimeric Pair:

### D-Glucose and D-Mannose:

Glucose: The hydroxyl group (—OH) at C-2 is on the right.

Mannose: The hydroxyl group (—OH) at C-2 is on the left.

Thus, an epimer is essentially a sugar that is identical to another sugar except for the arrangement of groups around one chiral carbon.

While galactose is a monosaccharide like tagatose and allulose, it does not have a lower sweetness, higher caloric content, and greater impact on blood sugar levels. Mannose can be used in conjunction with or substituted for galactose.

Galactose lacks the low-calorie and low-glycemic benefits that make tagatose and allulose attractive as sugar substitutes. This may, however, slow the fermentation process for kombucha, so Galactose can be combined or solely utilized as an added substrate for kombucha manufacturing to yield a lower alcohol (ethanol) content for any particular fermentation period.

In a preferred embodiment, the added monosaccharides consist essentially of allulose, tagatose and combinations thereof. The use of these particular monosaccharides reduces the alcohol content of the kombucha produced, yield a nice flavor, enable pro-biotic activity in vivo, and impart shelf stability.

Allulose and tagatose are sugars that have gained attention due to their low-calorie content, potential health benefits, and close resemblance to regular sugar in taste and texture. They are both classified as monosaccharides, but their metabolism in the body is different from common sugars like glucose and fructose. Below is a detailed breakdown of their qualities, including sweetness levels and caloric content:

Allulose (D-psicose) is about 70% as sweet as sucrose (table sugar). Allulose provides only about 0.2 to 0.4 calories per gram, which is significantly lower than sucrose (which provides 4 calories per gram). Most of the allulose consumed is excreted from the body without being metabolized, which is why it contributes so few calories. Allulose is absorbed in the small intestine but is not metabolized for energy, meaning it doesn't raise blood glucose or insulin levels, making it an attractive option for people with diabetes or those following low-carb diets. Studies have shown potential health benefits of Allulose consumption, such as improved blood sugar regulation, fat reduction, and anti-inflammatory effects.

Tagatose is 92% as sweet as sucrose, so it is nearly as sweet as regular sugar. It also provides a clean, sweet taste with a slight cooling effect on the tongue, which is not overwhelming. Like allulose, it has no bitter aftertaste, making it suitable for use as a sugar substitute. Tagatose provides about 1.5 calories per gram, which is still much lower than the 4 calories per gram found in sucrose but higher than allulose. Around 20-30% of tagatose is absorbed in the small intestine, while the rest is fermented in the large intestine by gut bacteria, contributing to its lower caloric value. Tagatose has a minimal impact on blood glucose and insulin levels, making it a favorable choice for people with diabetes or those looking to manage their weight.

Tagatose is partially fermented by gut bacteria, and some studies suggest it may have prebiotic effects, promoting the growth of beneficial gut bacteria. Tagatose may also inhibit the digestion of certain carbohydrates, reducing postprandial blood sugar spikes.

Table 2 below is a Tagatose and Allulose Comparison Summary

TABLE 2

| Quality | Allulose | Tagatose |
|---|---|---|
| Sweetness (vs. Sucrose) | 70% | 92% |
| Calories per Gram | 0.2-0.4 kcal | 1.5 kcal |

Allulose has a relatively low caloric content and minimal glycemic impact. Therefore, allulose is ideal for low-calorie, low-carb, and keto-friendly products.

Tagatose has a high sweetness and prebiotic potential, tagatose is used in diabetic-friendly foods and beverages such as the present invention. Both allulose and tagatose offer substantial benefits as sugar alternatives in reducing calories, controlling blood sugar, and providing functionality similar to sugar in various food applications.

SCOBY (Symbiotic Culture of Bacteria and Yeast): The kombucha fermentation process is driven by a combination of yeast and acetic acid bacteria. Common yeast strains include Saccharomyces cerevisiae, while bacteria such as Acetobacter are involved in converting ethanol to acetic acid.

There are various possible fermentations processes that can be employed according to the present invention. Below are two example fermentation processes.

### Example 1

1. Prepare a botanical extract from herbs, fruits, teas, or other plant materials. The extract is sweetened with the selected rare sugar(s) at the desired concentration, which may range from 5 mg/mL to 500 mg/mL.
   Inoculate the sweetened botanical extract is inoculated with SCOBY or a similar culture to create a fermentable mixture.
2. Ferment the mixture at room temperature (typically 68-78° F.) for 7-14 days, or until the desired flavor and acidity are achieved. During this period, the rare sugars are metabolized more slowly by the yeast, resulting in limited alcohol production.
3. An optional secondary fermentation can add flavor. For added flavor, the fermented beverage can undergo a secondary fermentation with selected fruits, herbs, or additional rare sugars.

### Example 2

1. Prepare a botanical extract from herbs, fruits, teas, or other plant materials. The extract is sweetened with the selected rare sugar(s) at the desired concentration, which may range from 5 mg/mL to 500 mg/mL.
2. Inoculate the sweetened botanical extract is inoculated with SCOBY or a similar culture to create a fermentable mixture.
3. Ferment the mixture at room temperature (typically 68-78° F.) for 7-14 days, or until the desired flavor and acidity are achieved. During this period, the rare sugars are metabolized more slowly by the yeast, resulting in limited alcohol production.
4. An optional secondary fermentation can add flavor or bioactivity. For added flavor, the fermented beverage can undergo a secondary fermentation with selected fruits, herbs, or additional rare sugars.
5. For bioactivity, the secondary fermentation can incorporate kratom, an extract from Mitragyna speciosa leaves or derived alkaloids (such as mitragynine and 7-hydroxymitragynine).

In various embodiments of the invention, kratom is added to the botanical extract prior to inoculation, during fermentation, or post-fermentation. The Kratom concentration may range from 0.1% to 5% by weight, adjusted to achieve desired effects while maintaining low alcohol levels. This integration leverages the low-alcohol fermentation enabled by allulose to produce a stable, probiotic-enhanced Kratom-infused kombucha. Kratom in combination with kombucha can mitigate the desire for alcohol in an alcoholic.

The use of monosaccharides such as allulose and its analogues (e.g., tagatose, psicose epimers, and other epimers) ensures that yeast fermentation is restricted, preventing excessive ethanol formation while allowing bacterial conversion to acetic acid. When combined with kratom, the product offers potential synergistic benefits, such as enhanced relaxation or wellness properties, without compromising the non-alcoholic nature of the beverage.

What is claimed is:

1. A method for producing a fermented beverage with reduced alcohol content, comprising:
preparing a base comprising brewed tea;
adding allulose or an analogue thereof as a primary sweetener at a concentration of 5 mg/mL to 500 mg/Ml;
inoculating the base with a symbiotic culture of bacteria and yeast (SCOBY); and
fermenting the inoculated base under conditions sufficient to produce a beverage with an alcohol content of less than 0.5% by volume.

2. The method of claim 1, wherein the analogue of allulose is selected from the group consisting of tagatose, sorbinose, psicose epimers, trehalose, isomaltulose, and combinations thereof.

3. The method of claim 1, further comprising adding a Kratom extract or Kratom-derived alkaloids to the base before, during, or after fermentation.

4. The method of claim 3, wherein the Kratom extract is derived from Mitragyna speciosa and is added at a concentration of 0.1% to 5% by weight.

5. The method of claim 1, wherein the fermentation is conducted at a temperature of 68° F. to 78° F. for 7 to 14 days.

6. A fermented beverage produced by the method of claim 1.

7. The fermented beverage of claim 6, further comprising Kratom alkaloids, wherein the beverage has a probiotic content and an alcohol content of less than 0.5% by volume.

8. The fermented beverage of claim 6, wherein the beverage has a caloric content of less than 2 kcal per gram from the sweetener and a low glycemic index.

9. A composition comprising:
providing a fermented tea base produced using SCOBY;
adding allulose or an analogue thereof;
adding kratom extract or alkaloids; and
wherein the composition has an alcohol content of less than 0.5% by volume and exhibits prebiotic properties.

10. The composition of claim 9, wherein the allulose or analogue thereof provides sweetness equivalent to 70% to 92% of sucrose while contributing 0.2 to 1.5 calories per gram.

11. The composition of claim 9, wherein the fermented tea base comprises acetic acid and live probiotic cultures selected from the group consisting of Saccharomyces cerevisiae, Acetobacter species, and combinations thereof.

12. The composition of claim 9, wherein the Kratom extract or alkaloids comprise mitragynine, 7-hydroxymitragynine, or combinations thereof.

13. The composition of claim 9, wherein the composition has a pH between 2.5 and 4.5 and is stable at room temperature for at least 6 months without significant increase in alcohol content.

14. The composition of claim 9, further comprising additional botanical extracts selected from the group consisting of herbs, fruits, and teas, wherein the allulose or analogue thereof constitutes at least 50% of the total fermentable sugars.

15. The composition of claim 9, wherein the analogue is tagatose, and the composition exhibits enhanced prebiotic effects promoting gut health.

16. A non-alcoholic fermented beverage composition comprising:
a tea base fermented with SCOBY;
allulose at a concentration of 5 mg/mL to 500 mg/mL;
kratom-derived alkaloids at 0.1% to 5% by weight; and
wherein the beverage has an ethanol content of less than 0.5% by volume, a caloric content from sweeteners of less than 1 kcal per gram, and contains live probiotics.

17. The composition of claim 16, further comprising one or more rare sugar analogues selected from tagatose, sorbinose, arabinose, trehalose, and isomaltulose.

18. The composition of claim 16, wherein the tea base is derived from black tea, green tea, Kratom tea, or combinations thereof.

19. The composition of claim 16, having a glycemic index lower than that of a comparable beverage sweetened with sucrose.

20. The composition of claim 16, wherein the composition is carbonated, bottled, and flavored with natural fruit or herb extracts.
